(19) United States
(12) Patent Application Publication
(10) Pub. No.: US 2005/0266392 A1
(43) Pub. Date: Dec. 1, 2005

(54) Central control system of specimen preservation devices

(75) Inventors: Yuichi Tamaoki, Ora-gun (JP); Atsushi Nakao, Neyagawa-shi (JP)

Correspondence Address: ARMSTRONG, KRATZ, QUINTOS, HANSON & BROOKS, LLP, 1725 K STREET, NW, SUITE 1000, WASHINGTON, DC, 20006, US

(73) Assignees: SANYO ELECTRIC CO., LTD., Moriguchi-shi (JP); SANYO ELECTRIC BIOMEDICAL CO., LTD., Moriguchi-shi (JP)

(21) Appl. No.: 11/114,242

(22) Filed: Apr. 26, 2005

(30) Foreign Application Priority Data
Apr. 26, 2004 (JP) 129201/2004

(51) Int. Cl. A01N 1/02; G06F 19/00; G01N 33/48; G01N 33/50

(52) U.S. Cl. 435/2; 702/19; 235/375

(57) ABSTRACT

An object is to provide electrical locks in a plurality of culture devices represented by incubators and in a plurality of preservation devices represented by chemical coolers, in order to build a system based on the idea of one chamber one donor; and to provide a central control system wherein the one chamber one donor is clearly indicated by a bar code and it is thus difficult to cause contamination of specimens corresponding to donors due to a human operational error. In summary, the central control system comprises a bar code scanner connected to a personal computer which centrally controls electrical locks of a plurality of specimen preservation devices with electrical locks to preserve target specimens in regenerative medicine, and driver units which open/shut the electrical locks of the respective preservation devices, wherein the specimen preservation device preserves a culture bottle or a culture media preservation bottle containing a specimen of one donor to which a bar code label is affixed, and the driver unit only opens/shuts the electrical lock of the specimen preservation device storing the specimen to which the same label as the bar code read by the scanner is affixed, in accordance with an instruction from the personal computer.

### BACKGROUND OF THE INVENTION

1. Field of the Invention

The present invention relates to a central control system of specimen preservation devices which collectively controls a plurality of sample preservation apparatuses (these are generically called specimen preservation devices), such as a cabinet for biological hazard measures which forms a work space maintained in a clean environment and which prevents microorganisms, bacteria and the like from leaking out, a plurality of culture apparatuses comprising carbon dioxide (CO₂) incubators and multi-gas incubators which cultivate a culture (sample) such as cells and microorganisms, and a chemical cooler which cool-preserves chemicals and blood.

2. Description of the Related Art

A cabinet for biological hazard measures (heretofore referred to as a safety cabinet, and hereinafter simply referred to as a cabinet) forms a work space maintained in a clean (germfree) environment where samples such as hazardous microorganisms, bacteria and the like are handled, and an incubator (culture apparatus) maintains a constant inside temperature (inside the culture chamber) and a constant CO₂ concentration and keeps the inside in a germfree condition so as to cultivate a culture (sample) such as cells and microorganisms to be cultured, so that it is necessary to periodically sterilize the inside. Samples taken from the cabinet may be stored per sample independently and separately in a plurality of cultures apparatuses and chemical coolers (i.e., sample preservation apparatuses), in order to achieve specimen preservation.

On the other hand, Japanese Patent Publication Laid-open No. 2002-373372 (Document 1) has shown an automatic vending apparatus comprising a processing space and a processing machine provided in a main body equipped with a cooler, and an open/close door provided at an entry/exit of the processing space, wherein an automatic locking apparatus is provided which automatically locks the door when the open/close door is closed.

Moreover, Japanese Patent Publication Laid-open No. 9-237538 (Document 2) has shown a counter system which performs central information control over a plurality of counters through a telecommunications network, and this system comprises a scanner to read image information and a recognition applied procedure processing unit to identify and read numbers and characters.

First, it will be assumed that the vending apparatus as shown in Document 1 is replaced with a culture apparatus having an electrical lock. That is, whenever a sample (specimen) is moved between the cabinet and the culture device or the chemical cooler, a manual operation is required, and this human intervention has raised concern that the specimen may be contaminated with miscellaneous germs.

There have recently been a cross contamination problem in good manufacturing practice (GMP) facilities, and a possibility of so-called contamination in which substances and gases contaminated with miscellaneous germs are leaked from the work space to the outside of the cabinet when the specimen is brought in and out of the cabinet. It is furthermore necessary to increase, for safety improvement, air cleanness of a room where the cabinet and the culture device or the chemical cooler are placed, and this has posed a problem of increased equipment expenses due to, for example, addition of a cleaning apparatus into the room. In addition, it has been typically recommended to manage the specimens such that one room contains a specimen of a certain person (an idea of so-called one room one donor) in principle, but such a scheme has a disadvantage that it necessitates a plurality of rooms when a plurality of donors is managed, which results in a significantly large facility for a system, and when this system is industrialized, it requires such a high cost that manufactures are not able to sell or install the system due to economical infeasibility.

On the other hand, assuming that the counter system as shown in Document 2 is replaced with a central control system, a personal computer which is the most general form of a central control system is disposed in one room, and in a room different from the one where the personal computer is disposed, there are arranged a cabinet connected to the personal computer by a communication line, a plurality of incubators, and a plurality of chemical coolers. In this case, the personal computer centrally controls the cabinet, the plurality of incubators and the plurality of chemical coolers, independently from each other. When a user of the central control system operates the personal computer in the one room in order to perform operations of storing and taking out the specimen for one control device (particularly the same thing), it is also possible to dispose a plurality of specimens in the incubator and the chemical coolers usually having a plurality of shelves. This might cause the mix-up of specimens; the specimens of different donors are stored in the same device due to a human operational error, and the specimen of a desired person cannot be stored in a desired device, and it has been impossible to prevent this error.

### SUMMARY OF THE INVENTION

It is an object of the present invention, in such a central control system, to provide electrical locks in a plurality of culture devices represented by incubators, and in a plurality of preservation devices such as coolers represented by chemical coolers and freezers represented by ultra-deep freezers (these are generically called a plurality of specimen preservation devices), in order to build a system based on the idea of one chamber one donor, and to make it difficult to cause contamination of specimens corresponding to donors due to a human operational error, while the one chamber one donor is clearly indicated by a bar code. It is another object thereof to provide a central control system in which the specimen preservation device to be unlocked or locked specified by a personal computer is visually recognized by a user for convenience of central control, such that signal communications can be performed to prevent the contamination of the specimens especially when the specimen preservation device is disposed in a room different from the one where the personal computer is disposed.

A central control system (1) of the present invention comprises a plurality of specimen preservation devices (31, 33) with electrical locks (21, 23) which preserve target specimens in regenerative medicine (tissue engineering); a personal computer (5) which centrally controls the electrical locks (21, 23) of the respective preservation devices; a bar code reader (6) and a bar code issuer (7) connected to the personal computer (5); and driver units (2,3) which are located between the respective preservation devices (31, 33) and the personal computer (5) and which open/shut the electrical locks (21, 23) of the respective preservation devices, wherein each specimen preservation device (31, 33) preserves a culture bottle (10) or a culture media preservation bottle (11) containing a specimen of one donor to which a bar code label (15) is affixed, and the driver unit (2,3) opens/shuts, in accordance with an instruction from the personal computer (5), the electrical lock (21, 23) of the specimen preservation device (31, 33) storing the specimen to which the same bar code label as the bar code (15) read by the bar code reader (6) is affixed.

According to this invention, the specimen preservation device storing the specimen to which the same bar code label as the bar code (15) read by the bar code reader (6) is affixed is selected from the plurality of specimen preservation devices (31, 33) by the driver units (2, 3) in accordance with the instruction from the personal computer (5), such that the electrical lock (21, 23) of the selected preservation device can be independently open-shut-controlled and managed, such that the specimen of one donor can be stored and preserved in each of the specimen preservation devices (31, 33), and such that a structure of one donor and one camber can be built.

Furthermore, a central control system (1) of the present invention comprises a plurality of specimen preservation devices (31, 33) with electrical locks (21, 23) which preserve target specimens in regenerative medicine; a personal computer (5) which is connected to the respective preservation devices by communication lines (41, 43) and which centrally controls the electrical locks (21, 23) of the respective preservation devices; a bar code reader (6) and a bar code issuer (7) connected to the personal computer (5); and driver units (2,3) which are located between the respective preservation devices (31, 33) and the personal computer (5) and which open/shut the electrical locks (21, 23) of the respective preservation devices, wherein a bar code (15) of one donor issued by the bar code issuer (7) is affixed to a process instruction (12) corresponding to a culture bottle (10) or a culture media preservation bottle (11) containing the specimen of one donor, and in accordance with a result of reading the affixed bar code (15) of the process instruction (12) by the bar code reader (6), the driver unit (2, 3) only opens/shuts the electrical lock (21, 23) of the specimen preservation device (31, 33) storing the specimen to which the bar code is affixed.

According to this invention, the bar code (15) affixed to the process instruction (12) is read by the bar code reader (6), and the electrical lock (21, 23) of the specimen preservation device storing the culture bottle (10) or the culture media preservation bottle (11) to which the same bar code corresponding to the reading result is affixed can be opened/shut by the driver units (2, 3), thereby building the state of one donor one chamber. Further, it is possible to build a structure wherein if even a single electrical lock of the specimen preservation device is opened, the other electrical locks of the other specimen preservation devices can be locked into a shut state, thereby making it possible to prevent the mix-up of specimens due to a human operational error and to improve the safety in the field of regenerative medicine.

Furthermore, in the central control system (1) of the present invention, the specimen preservation device(s) is (are) a culture apparatus (31), a chemical cooler (33) and/or an ultra-deep freezer (35).

According to this invention, the culture apparatus (31), the chemical cooler (33) and/or the ultra-deep freezer (35) is (are) selected as the specimen preservation device(s) such that the central control system can be built which is capable of long-term preservation of the target specimen as well as the culture thereof.

Furthermore, in the central control system (1) of the present invention, the specimen preservation device is provided with an indicator portion (41, 43).

According to this invention, the specimen preservation device whose electrical lock (21, 23) has been opened can only be indicated by the indicator portion (41, 43) in an indicating state different from that of all the other specimen preservation devices whose electrical locks are shut, thus making it more convenient for the user.

Furthermore, in the central control system (1) of the present invention, the driver unit (2, 3) controls the indicator portion (41, 43) of the specimen preservation device whose electrical lock is opened into an indicating state different from that of the indicator portion (41, 43) of the other specimen preservation devices.

According to this invention, the indicator portion (41, 43) of the specimen preservation device whose electrical lock is opened is controlled to be in an indicating state different from that of the indicator portions of the other specimen preservation devices, such that it is possible to visually recognize the specimen preservation device (31, 33) whose electrical lock has been opened, that is, whose door can be opened, such that it is possible to find out the device which the specimen should be brought in or out of, and such that it is possible to prevent errors in taking in and out the specimen and, that is, errors due to human operation.

Furthermore, a central control system (1) of the present invention comprises a plurality of specimen preservation devices (31, 33) with electrical locks (21, 23) which preserve target specimens in regenerative medicine, a personal computer (5) which centrally controls the electrical locks (21, 23) of the respective preservation devices, a bar code reader (6) and a bar code issuer (7) connected to the personal computer (5), and driver units (2, 3) which are located between the respective preservation devices (31, 33) and the personal computer (5) and which open/shut the electrical locks (21, 23) of the respective preservation devices, wherein the bar code issuer (7) comprises an input portion to input information regarding the specimen, and a printer portion which converts the information into a bar code to print the bar code.

According to this invention, the information regarding the specimen can be input in the input portion to print the bar code (15) corresponding to the information; the printed bar code (15) can be affixed to the corresponding culture bottle (10) or culture media preservation bottle (11); the target specimen in the regenerative medicine can be identified and managed by the bar code (15); the specimen preservation device is identified which stores the specimen to which the same bar code label as the bar code (15) corresponding to the input information regarding the specimen is affixed; and the electrical lock (21, 23) of the identified specimen preservation device is opened/shut. In this way, the specimen of one donor can be stored and preserved in each of the specimen preservation devices (31, 33), and the electrical lock (21, 23) of the desired specimen preservation device can be remotely operated via the driver unit (2, 3), thereby improving the use and safety in the central control system.

Furthermore, the central control system (1) of the present invention comprises a plurality of bar codes (15) corresponding to a certain donor which are issued by the bar code issuer (7) and which correspond to the corresponding culture bottle (10) or culture media preservation bottle (11) containing the specimen of the certain donor and which are affixed to the corresponding process instruction (12).

According to this invention, a plurality of (here at least two) identical bar codes (15) is prepared, and they can be affixed to the culture bottle (10) or the culture media preservation bottle (11) and to the process instruction (12), respectively, in a corresponding manner, so that the desired specimen can be easily managed.

Furthermore, a central control system (1) of the present invention comprises a plurality of specimen preservation devices (31, 33) with electrical locks (21, 23) which preserve target specimens in regenerative medicine, a personal computer (5) which is connected to the respective preservation devices by communication lines (41, 43) and which centrally controls the electrical locks (21, 23) and the indicator portions (41, 43) of the respective preservation devices, and driver units (2, 3) which are connected to the respective preservation devices (31, 33) by signal lines (48, 49) and which are connected to the personal computer (5) by communication lines (42, 44, 45) to be located between the respective preservation devices and the personal computer and which open/shut the electrical locks (21, 23) of the respective preservation devices.

According to this invention, the electrical locks (21, 23) of the respective specimen preservation devices (31, 33) can be centrally opened/shut by the personal computer (5) and the driver units (2, 3), and the electrical lock (21, 23) of the desired specimen preservation device can be centrally controlled.

Furthermore, the central control system (1) of the present invention comprises a bar code reader (6) connected to the personal computer (5).

According to this invention, the target specimen in the regenerative medicine can be identified and managed by the bar code (15).

Furthermore, in the central control system (1) of the present invention, the bar code issuer (7) connected to the personal computer (5) comprises an input portion to input information regarding the specimen, and a printer portion which converts the information into a bar code to print the bar code.

According to this invention, the information regarding the specimen can be input in the input portion to print the bar code (15) corresponding to the information, and the printed bar code (15) can be affixed to the corresponding culture bottle (10) or culture media preservation bottle (11) and to the process instruction (12), so that the specimen can be managed by the bar code.

Furthermore, in the central control system (1) of the present invention, the specimen preservation device (31, 33) is provided with an open/close sensor which detects opening and closing of the door, and the driver unit (2, 3) automatically shuts the electrical lock (21, 23) when the door is closed in accordance with a signal from the sensor.

According to this invention, the user closes the door whenever he brings the specimen in and out of the specimen preservation device (31, 33), so that an automatic shutting instruction can be given by the driver unit (2, 3) without the user instructing to shut the electrical lock (21, 23) when the door closed, which makes it possible to prevent an error in shutting the electrical lock (21, 23) due to human operation.

### BRIEF DESCRIPTION OF THE DRAWINGS

FIG. 1 is a configuration diagram of a central control system of the present invention;

FIG. 2 is a wiring-related view of a driver unit of the present invention;

FIG. 3 is a flowchart to set and release an electrical lock of the present invention;

FIG. 4 is a longitudinal sectional view of essential parts of the electrical lock of the present invention;

FIG. 5 are perspective views showing states of the mechanically set and released electrical lock of the present invention; and

FIG. 6 is a perspective view showing the state around the electrical lock when a door of a preservation device of the present invention is opened.

### DETAILED DESCRIPTION OF THE PREFERRED EMBODIMENT

Embodiments of the present invention will be described below in detail with reference to the drawings.

(Embodiment 1)

A central control system (in view of functionality, referred to as a mix-up prevention system) 1 of specimen preservation devices in a first embodiment of the present invention is disposed in a cell processing center situated in a building where regenerative medicine takes place such as an office at university or a hospital, and electrical locks are provided in a plurality of specimen preservation devices so as to build a system based on the idea of one chamber one donor, and the system makes it difficult to cause contamination of a specimen, while the one chamber one donor is clearly indicated by a bar code.

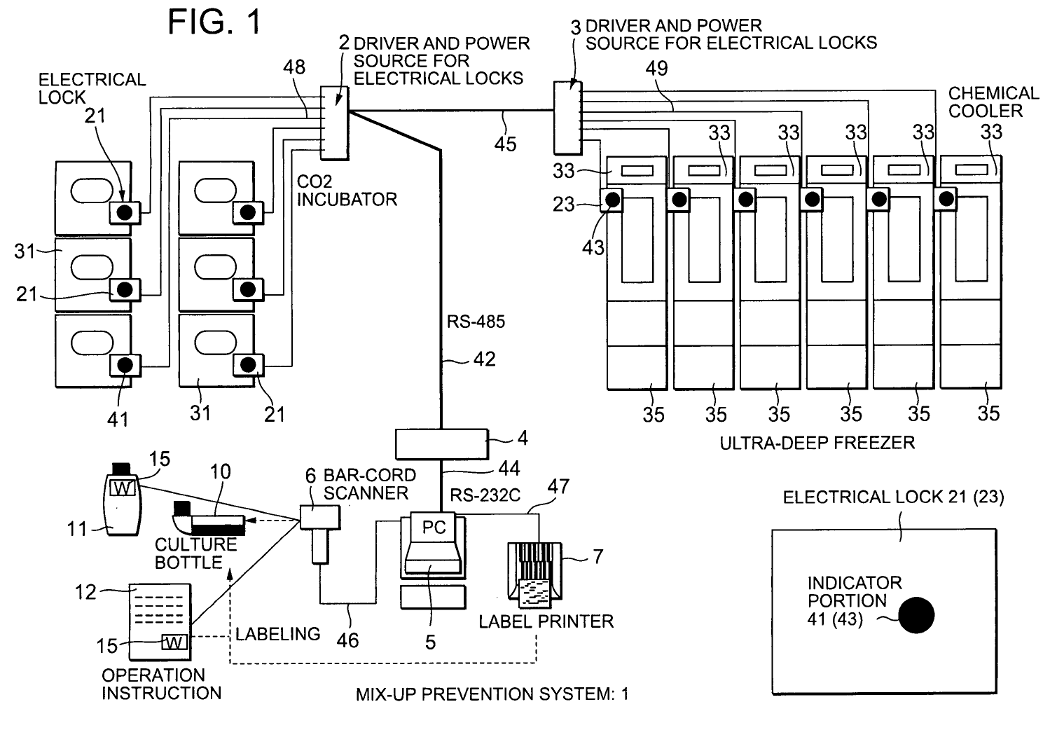

As shown in FIG. 1, the central control system comprises a plurality of specimen preservation devices 31, 33 with electrical locks 21, 23 which preserve target specimens in regenerative medicine (e.g., donor cells), a personal computer 5 for specimen management which is connected to the respective preservation devices by communication lines 42, 44, 45 and which centrally controls the electrical locks 21, 23 and indicator portions (41, 43) of the respective preservation devices, driver units 2, 3 which are connected to the respective preservation devices 31, 33 by signal lines 48, 49 and which are connected to the personal computer 5 by communication lines 42, 44, 45 to be located between the respective preservation devices and the personal computer and which open/shut the electrical locks 21, 23 of the respective preservation devices, and a bar code scanner 6 as a bar code reader and a bar code issuer 7 connected to the personal computer 5 by signal lines 46, 47.

The specimen preservation devices 3 include a culture apparatus 31 represented by an incubator, a chemical cooler 33 comprising a cooling apparatus, a cabinet for biological hazard measures, an ultra-deep freezer 35, a constant temperature and humidity box, a sterilized box and the like, and in the present embodiment, the incubator 31, the chemical cooler 33 and the ultra-deep freezer 35 are adopted.

The incubator 31 forms a culture chamber therein by a heat insulating box main body having an opening at the front and by a heat insulating door which closes the opening in an openable and closable manner, and it maintains a constant temperature and CO₂ concentration inside (inside the culture chamber) and brings the inside in a germfree condition so as to cultivate a culture (sample) such as cells and microorganisms to be cultured, so that it is necessary to periodically sterilize the inside. Moreover, for the culture of the sample, a heater and its controller are provided to regulate the temperature inside the culture chamber (storeroom). The inside of the culture chamber is vertically divided by a plurality of shelves, and a humidification plate retaining water for humidification is disposed at the bottom of the culture chamber and heated by heating means disposed on the outer side of the bottom of an inner box made of a metal such as stainless steel in order to evaporate water. Behind the heat insulating door, a transparent inner door is disposed to open/close a front opening 2A.

The chemical cooler 33 forms a cooling chamber therein by a heat insulating box main body having an opening at the front and by a heat insulating door which closes the opening in an openable and closable manner, and circulates the air cooled by a cooler provided at the bottom into the cooling chamber to cool the inside of the cooling chamber to a desired temperature. In the present example, two heat insulating doors are disposed at the top and bottom, and the top door is provided with a transparent window so that the inside is visible.

Furthermore, the electrical lock 21 of the incubator 31 and the electrical lock 23 of the chemical cooler 33 are provided with indicator portions 41, 43, respectively, to indicate the open/shut state of the electrical locks, and the operating state and abnormal state of the preservation device. In this indicator portion 41, 43, specifically, an indication light such as an LED, a lamp or a liquid crystal is disposed. In the present embodiment, the lamp is adopted, and this lamp is turned on when the electrical lock is opened. It is to be noted that the present embodiment has illustrated the ultra-deep freezer 35 without an electrical lock, but the ultra-deep freezer with an electrical lock should preferably be selected.

The bar code issuer 7 comprises an input portion to input information regarding the specimen, and a printer portion which converts the information into a bar code to print the bar code, and a bar code (in the present embodiment, two bar codes) 15 of one donor issued by the bar code issuer 7 (in particular, the printer portion) is affixed to a process instruction 12 corresponding to a culture bottle 10 or a culture media preservation bottle 11 containing the specimen of one donor. The affixed bar code 15 of the process instruction is read by the bar code reader 6, and the centrally controlling personal computer 5 performs operational control such that the electrical lock 21, 23 of the specimen preservation device 31, 33 storing the specimen to which the read bar code is affixed is only opened/shut.

As described above in detail, according to the present mix-up prevention system 1, each of the electrical locks 21, 23 of the plurality of specimen preservation devices 31, 33 can be open-shut-controlled and managed independently by the personal computer 5, thereby making it possible to build a structure of one donor one chamber where the specimen of one donor is stored and preserved in each of the specimen preservation devices 31, 33.

Furthermore, the bar code 15 affixed to the process instruction 12 is read by the bar code scanner 6, and the electrical lock 21, 23 of the specimen preservation device storing the culture bottle 10 or the culture media preservation bottle 11 to which the same bar code corresponding to the reading result is affixed can only be opened/shut by the personal computer 5, thereby building the state of one donor one chamber. Further, it is possible to build a structure wherein if even a single electrical lock of the specimen preservation device is opened, the other electrical locks of the specimen preservation devices can be locked into a shut state, thereby making it possible to prevent the mix-up of specimens due to a human operational error and to improve the safety in the field of regenerative medicine.

Furthermore, the culture apparatus 31 and the chemical cooler 33 are selected as the specimen preservation devices such that the central control system can be built which is capable of long-term preservation of the target specimen as well as the culture thereof, and the personal computer 5 causes the indicator portions 41, 43 to only indicate the specimen preservation device whose electrical lock 21, 23 has been opened in an indicating state different from that of all the other specimen preservation devices whose electrical locks are shut, such that it is possible to visually recognize the specimen preservation device 31, 33 whose electrical lock has been opened, that is, whose door can be opened, such that it is possible to find out the device which the specimen should be brought in or out of, and such that it is possible to prevent errors in taking in and out the specimen, that is, errors due to human operation.

The information regarding the specimen can be input in the input portion to print the bar code 15 corresponding to the information, and the printed bar code 15 can be affixed to the corresponding culture bottle 10 or culture media preservation bottle 11, and the target specimen in the regenerative medicine can be identified and managed by the bar code 15, thereby improving the use and safety in the central control system. Further, a plurality of (here at least two) identical bar codes (15) is prepared, and they can be affixed to the culture bottle 10 or the culture media preservation bottle 11 and to the process instruction 12, respectively, in a corresponding manner, so that the desired specimen can be easily managed. Still further, the user closes the door whenever he brings the specimen in and out of the specimen preservation device 31, 33, so that an automatic shutting instruction can be given by the personal computer 5 without the user instructing to shut the electrical lock (21, 23) when the door is closed, which makes it possible to prevent an error in shutting the electrical lock 21, 23 due to human operation.

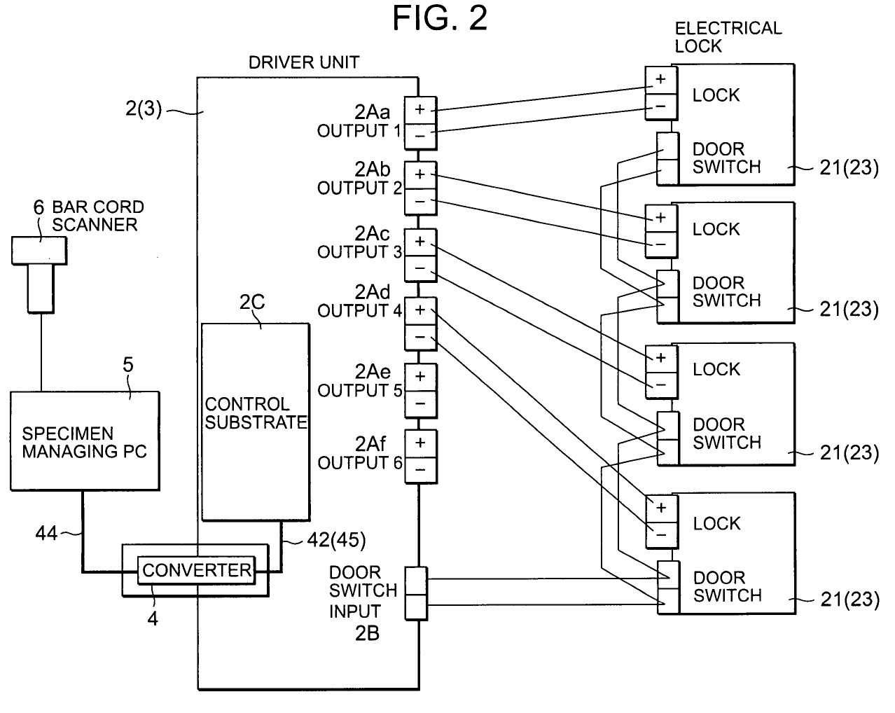

Next, the input and output of signals to and from the driver units 2, 3 will be described with reference to a block diagram of FIG. 2. The personal computer 5 and a converter 4 are connected by the signal line 44, and the converter 4 and the driver unit 2 (3) (in particular, a control substrate 2C) are connected by the signal line 42. The converter 4 converts a parallel signal of a RS-232C port to a communication signal of a RS-485 port, and substantially up to 31 driver units can be connected to this RS-485 port. Each driver unit has a plurality of (here six) output terminals 1 to 6 (2Aa, 2Ab, 2Ac, 2Ad, 2Ae, 2Af) and an input terminal 2B, and each output terminal is connected to a lock terminal of the electrical lock 21 (23) via the signal line 48 (49).

Furthermore, door switch terminals of the electrical locks 21 (23) are connected in parallel, and the door switch terminal of the single electrical lock is connected to the input terminal 2B of the driver unit 2 (3). Owing to this parallel connection, when one of the electrical locks 21 (23) is opened and the door is opened, a door switch detects it and outputs a signal, and this signal is input to the input terminal 2B of the driver unit. Up to 31 driver units can be connected to the communication line 44 (specifically, the RS-485 port), so that, considering that one driver unit has six output ports, 6×31=186 electrical locks at the maximum number can be connected to the communication line 44.

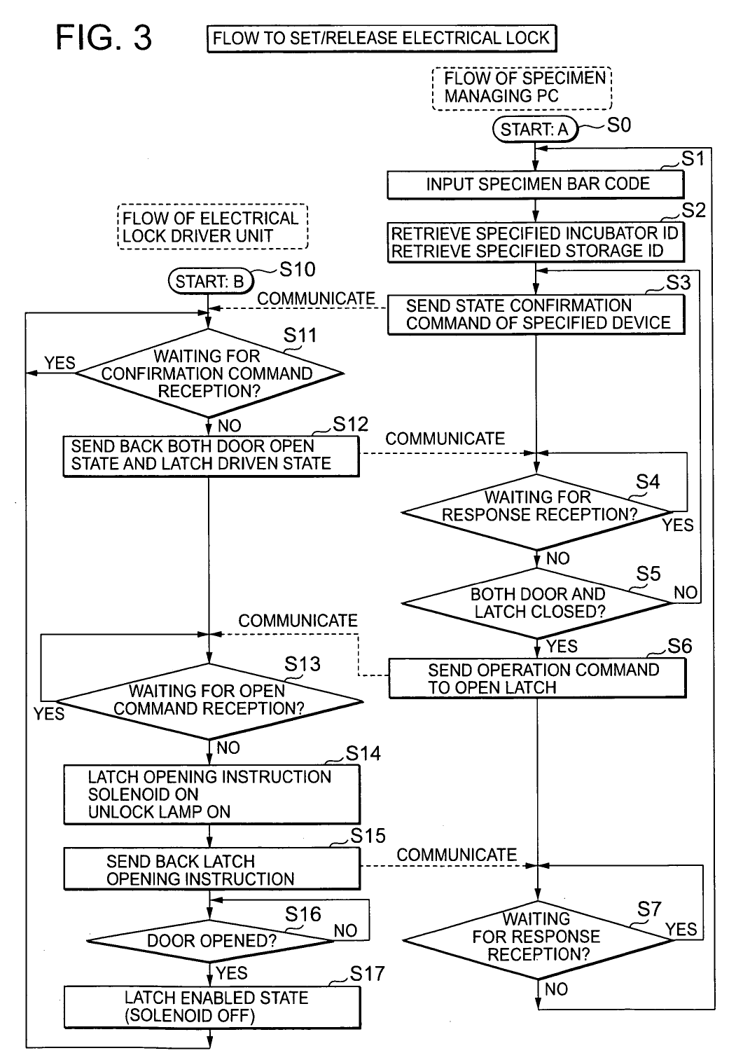

Now, operations of setting and releasing the electrical lock 21, 23 will be described separately on a personal computer side and a driver side, with reference to a flowchart of FIG. 3. First, the operation on the personal computer side will be described. Starting at step S0, a bar code is input at step S1, in order to identify the preservation device storing the specimen to be unblocked. The bar code may be input by manually operating the input portion or may be automatically input in such a manner that the bar code scanner 6 reads the bar code of the process instruction 12. In accordance with the bar code input at this step S1, an ID of the preservation device 31, 33, 35 preserving the bottle 10 or 11 to which the bar code is affixed is retrieved (identified) at step S2. In accordance with the retrieval result, the personal computer 5 outputs, via the communication lines 44 and 42, a signal (confirmation command) instructing to confirm the open/close state of the door of a particular device at step S3 in order to release the electrical lock of the device (i.e., the particular device) corresponding to the retrieved ID, thus moving to step S4.

Step S4 represents a state to wait for a state signal (response reception) regarding the door and a latch from the driver unit side; the waiting state continues until the state signal is input via the communication lines 42 and 44. If the state signal is input, step S5 judges whether both the door and the latch are in a closed state; if both the door and the latch are not in the closed state (i.e., if both of them are in the open state or if one of them is in the closed state), it returns to step S3; and if both the door and the latch are in the closed state, an open signal (operation command) to open the latch is output at step S6, thus moving to step S7.

Step S7 represents a state to wait for an unlock signal (response reception), meaning to wait for an operational instruction indicating that a signal of unlatching is output to be returned from the driver unit side via the communication lines 42 and 44, and the waiting state continues until the state signal is input via the communication lines 42 and 44, and when the unlock signal is input, it returns to step S1.

Next, the operation on the driver side will be described. Starting at step S10, the driver enters a state to wait for the confirmation command at step S11, and continues the waiting state until the confirmation command is input via the communication lines 42 and 44, and if the confirmation command is input, the states of the door and the latch are judged (whether they are opened or closed) at step S12, and the state confirmation result of the door and the latch is sent to the side of the personal computer 5 via the communication lines 42 and 44, thus moving to step S13.

At step S13, an open command to open the single (solely selected) latch also enters the waiting state, and the waiting state continues until the open command is input via the communication lines 42 and 44, and when the open command is input, an open operation signal (specifically, a signal which conducts a solenoid for unlatching and which turns on an unlock lamp as the indicator portion of the electrical lock 21 (23) to be released) of the latch is output at step S14. At next step S15, the operational instruction indicating that the signal of opening the latch has been output is returned via the communication lines 42 and 44, thus moving to step S16.

At step S16, a judgment is made on whether or not the door of the preservation device has been opened by the user, in accordance with an instruction to open the latch, and this judgment continues until the door is opened. When the door switch confirms that the door has been opened, a signal (i.e., a signal which stops conducting the solenoid for latching and which turns on the unlock lamp as the indicator portion of the electrical lock 21 (23) that has been opened) is output at step S17 to indicate that the latch is enabled, thus returning to step S11.

As described above, operations to confirm the signal or command are performed between the personal computer 5 and the driver unit 2 (3) via the communication lines 44 and 42 in the waiting state in the flowchart, and the signals or commands are successively exchanged between independent control systems.

Furthermore, the indicator portion 41, 43 of the specimen preservation device whose electrical lock 21 (23) is opened is controlled to be in the indicating state different from that of the indicator portions of the other specimen preservation devices, such that it is possible to visually recognize the specimen preservation device 31, 33 whose electrical lock has been opened, that is, whose door can be opened, such that it is possible to find out the device which the specimen should be brought in or out of, and such that it is possible to prevent errors in taking in and out the specimen, that is, errors due to human operation.

Moreover, the target specimen in the regenerative medicine can be identified and managed by the bar code 15; the specimen preservation device is identified which stores the specimen to which the same bar code label as the bar code 15 corresponding to the input information regarding the specimen is affixed; and the electrical lock 21, 23 of the identified specimen preservation device is opened/shut. In this way, the specimen of one donor can be stored and preserved in each of the specimen preservation devices 31, 33, and the electrical lock 21, 23 of the desired specimen preservation device can be remotely operated via the driver unit 2, 3, thereby improving the use and safety in the central control system.

As described above in detail, according to the present mix-up prevention system 1 as the first embodiment, each of the electrical locks 21, 23 of the plurality of specimen preservation devices 31, 33 can be open-shut-controlled and managed independently by the personal computer 5, thereby making it possible to build the structure of one donor one chamber where the specimen of one donor is stored and preserved in each of the specimen preservation devices 31, 33.

Furthermore, the bar code 15 affixed to the process instruction 12 is read by the bar code scanner 6, and the personal computer 5 can only open/shut the electrical lock 21, 23 of the specimen preservation device storing the culture bottle 10 or the culture media preservation bottle 11 to which the same bar code corresponding to the reading result is affixed, thereby building the state of one donor one chamber. Further, it is possible to build a structure wherein if even a single electrical lock of the specimen preservation device is opened, the other electrical locks of the specimen preservation devices can be locked into a shut state, thereby making it possible to prevent the mix-up of specimens due to a human operational error and to improve the safety in the field of regenerative medicine.

(Embodiment 2)

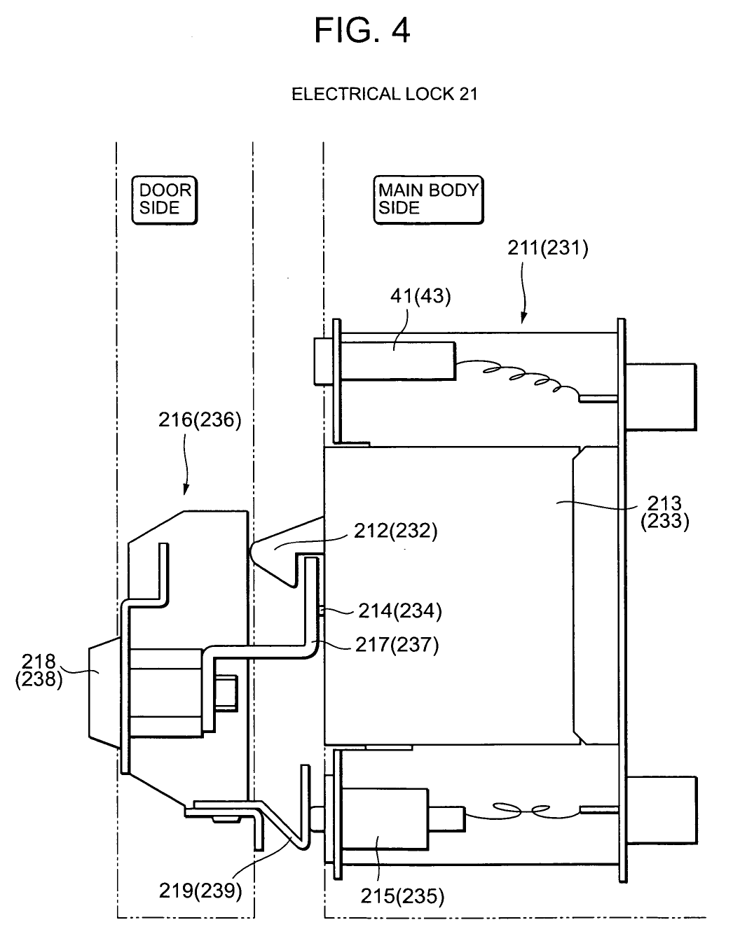

The structure of the electrical lock 21 (23) will be described as a second embodiment of the present invention referring to FIG. 4 to FIG. 6. In each drawing, the electrical lock 21 (23) comprises a lock main body 211 (231) fixed to the main body side of the preservation device 31, 33, 35, and a lock receiving portion 216 (236) fixed to the door (in particular, heat insulating door) side of the preservation device 31, 33, 35.

The lock main body 211 (231) comprises a lock drive portion 213 (233) having a claw member 212 (232) to switch locking/unlocking of the door, the unlock lamp 41 (43) as the indicator portion which lights when the claw member 212 (232) is unlocked to indicate that the door can be opened/closed, an open/shut regulating member 214 (234) which regulates the operation of the claw member 212 (232) when it is pushed in by a key member described later, and a door switch 215 (235) which comes in and out in accordance with the open/shut operation of the door and which detects the open/close state of the door. The lock drive portion 213 always urges the claw member in a closing direction by an urging member such as a spring so as to bring the claw member 212 into a lock position when it is not conducting, and is provided with an electromagnetic coil which operates to cancel the urge of the claw member 212 against the pushing force of the urging member during conduction.

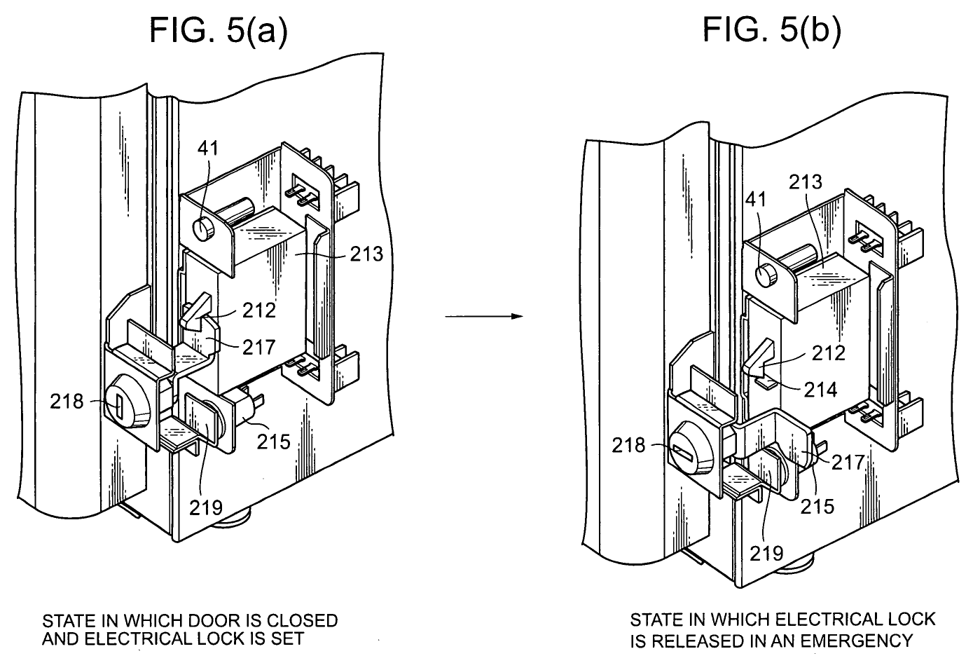

The lock receiving portion 216 (236) comprises a key member 217 (237) which normally engages with the claw member 212 (232) to constitute a lock state and which is manually turned in an emergency to be unengaged with the claw member 212 (232), and a press member 219 (239) which lets in/out the door switch 215 (235) synchronously with the open/close operation of the door. 218 is a key hole to manually turn the key member 217 (237) when the electrical lock 21 (23) cannot be released for any reason such as a power failure or a breakdown. As shown in FIG. 5(a), normally, the door is closed and the electrical lock is set, and as shown in FIG. 5(b), in an emergency, an unshown master key is inserted into the key hole 218 and then revolved in one direction (here rightward), so that synchronously with this revolution, the key member 217 revolves rightward and cancels the engagement with the claw member 212, thereby unlocking the door.

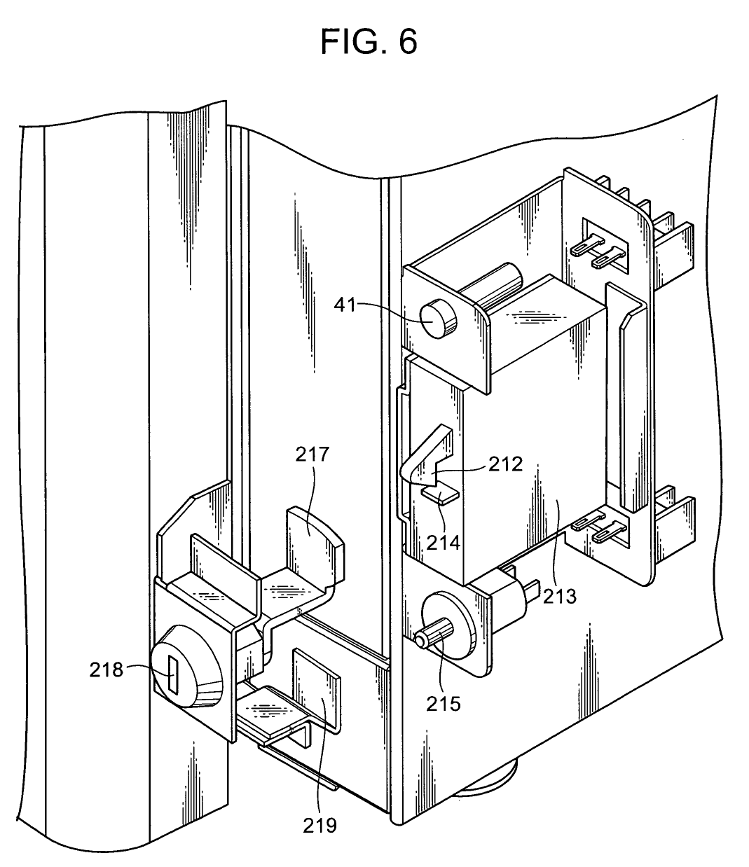

It is to be noted that, as shown in FIG. 5(a), if the lock drive portion 213 is conducted in a normal state, the claw member 212 and the key member 217 are unengaged, so that the door can be freely opened if the door is operated to be opened, as shown in FIG. 6(a). At this time, the door switch 215 is projecting and a signal is output to indicate that the door has been opened, and the open/shut regulating member 214 is also projecting and does not regulate the operation of the claw member 212. Therefore, in the case where the door is closed, if an edge of the key member 217 collides with the claw member 212 in accordance with the closing operation, the claw member 212 which has become unregulated due to the force of closing is pushed upward in a revolving manner, and if the door is completely closed, the key member 217 can come into a rear position of the claw member 212. In this state, because electricity is turned on, the urging force of the claw member 212 does not act and the engagement with the key member 217 has been cancelled. Then, if the conduction of the lock drive portion 213 is stopped, the claw member 212 is activated by the urging force to complete the engagement with the key member 217, which results in the lock state and regulates the operation of opening the door.

As described above in detail, if the electrical lock as the second embodiment is adopted, the master key can be inserted into the key hole 218 and then revolved such that the lock state of the electrical lock is manually cancelled regardless of whether the lock drive portion 213 is conducted, thereby making it possible to avoid unforeseen situations where the door of the preservation device cannot be opened in an emergency and providing the improved general-purpose central control system of the preservation devices.

1. A central control system of specimen preservation devices comprising: a plurality of specimen preservation devices with electrical locks which preserve target specimens in regenerative medicine; a personal computer which centrally controls the electrical locks of the respective preservation devices; a bar code reader and a bar code issuer connected to the personal computer; and driver units which are located between the respective preservation devices and the personal computer and which open/shut the electrical locks of the respective preservation devices, wherein each specimen preservation device preserves a culture bottle or a culture media preservation bottle containing a specimen of one donor to which a bar code label is affixed, and the driver unit opens/shuts, in accordance with an instruction from the personal computer, the electrical lock of the specimen preservation device storing the specimen to which the same bar code label as the bar code label read by the bar code reader is affixed.

2. A central control system of specimen preservation devices comprising: a plurality of specimen preservation devices with electrical locks which preserve target specimens in regenerative medicine; a personal computer which centrally controls the electrical locks of the respective preservation devices; a bar code reader and a bar code issuer connected to the personal computer; and driver units which are located between the respective preservation devices and the personal computer and which open/shut the electrical locks of the respective preservation devices, wherein a bar code of one donor issued by the bar code issuer is affixed to a process instruction corresponding to a culture bottle or a culture media preservation bottle containing the specimen of one donor, and the driver unit reads the affixed bar code of the process instruction with the bar code reader and opens/shuts the electrical lock of the specimen preservation device storing the specimen to which the bar code is affixed.

3. The central control system of the specimen preservation devices according to claim 2, wherein the specimen preservation device(s) is (are) a culture apparatus, a chemical cooler and/or an ultra-deep freezer.

4. The central control system of the specimen preservation devices according to claim 2, wherein the specimen preservation device is provided with an indicator portion.

5. The central control system of the specimen preservation devices according to claim 4, wherein the driver unit controls the indicator portion of the specimen preservation device whose electrical lock is opened into an indicating state different from that of the indicator portion of the other specimen preservation device.

6. A central control system of specimen preservation devices comprising: a plurality of specimen preservation devices with electrical locks which preserve target specimens in regenerative medicine; a personal computer which centrally controls the electrical locks of the respective preservation devices; a bar code reader and a bar code issuer connected to the personal computer; and driver units which are located between the respective preservation devices and the personal computer and which open/shut the electrical locks of the respective preservation devices, wherein the bar code issuer comprises an input portion to input information regarding the specimen, and a printer portion which converts the information into a bar code to print the bar code; and the personal computer identifies the specimen preservation device storing the specimen to which the same bar code label as the bar code corresponding to the information regarding the specimen input at the input portion is affixed, and causes the driver unit to open/shut the electrical lock of the identified specimen preservation device.

7. The central control system of the specimen preservation devices according to claim 6, comprising a plurality of bar codes corresponding to a certain donor which are issued by the bar code issuer and which correspond to the culture bottle or the culture media preservation bottle containing a specimen of the certain donor and which are affixed to the corresponding process instruction.

8. A central control system of specimen preservation devices comprising: a plurality of specimen preservation devices with electrical locks which preserve target specimens in regenerative medicine; a personal computer which centrally controls the electrical locks of the respective preservation devices; and driver units which are connected to the respective preservation devices by signal lines and which are connected to the personal computer by communication lines to be located between the respective preservation devices and the personal computer and which open/shut the electrical locks of the respective preservation devices.

9. The central control system of the specimen preservation devices according to claim 8, comprising a bar code reader connected to the personal computer.

10. The central control system of the specimen preservation devices according to claim 8, wherein the bar code issuer connected to the personal computer comprises an input portion to input information regarding the specimen, and a printer portion which converts the information into a bar code to print the bar code.

11. The central control system of the specimen preservation devices according to claim 9, wherein the specimen preservation device is provided with an open/close sensor which detects opening and closing of a door, and the driver unit automatically shuts the electrical lock when the door is closed in accordance with a signal from the sensor.
